(19) United States
(12) Patent Application Publication
(10) Pub. No.: US 2008/0082098 A1
(43) Pub. Date: Apr. 3, 2008

(54) ELECTRIC PROCESSING SYSTEM

(76) Inventors: Kazue TANAKA, Sagamihara-shi (JP); Takashi IRISAWA, Akishima-shi (JP); Takashi MIHORI, Akiruno-shi (JP)

Correspondence Address: OSTROLENK FABER GERB & SOFFEN, 1180 AVENUE OF THE AMERICAS, NEW YORK, NY, 100368403, US

(21) Appl. No.: 11/537,160

(22) Filed: Sep. 29, 2006

(51) Int. Cl. A61B 18/18

(52) U.S. Cl. 606/41

(57) ABSTRACT

There is provided an electric processing system which sequentially monitors a phase difference of intermittently output high-frequency powers in the case of performing feedback control with respect to a high-frequency power applied to bipolar type sealing forceps, reduces the high-frequency power and prolongs an application time at the time of occurrence of abnormal discharge (a spark) at distal ends, thereby terminating the abnormal discharge (extinguishing the spark) to carry out sealing processing.

### BACKGROUND OF THE INVENTION

1. Field of the Invention

The present invention relates to an electric processing system which performs a surgical procedure such as coagulation of a living tissue or sealing of a blood vessel for hemostasis by using a high-frequency power.

2. Description of the Related Art

In general, there is known an electric surgical apparatus which uses a high-frequency power to perform a surgical procedure such as incision, coagulation or hemostasis with respect to a living tissue as typified by an electric scalpel. As a similar apparatus, there are known sealing forceps which use a high-frequency power to hermetically close and weld a blood vessel or a fibrovascular bundle. For example, in U.S. Pat. No. 6,033,399 (U.S. patent application Ser. No. 08/8,385,458) is proposed a generator for an electric surgical apparatus (an electrosurgical generator) having an output control portion which performs feedback control by monitoring an impedance of a living tissue to desiccation the living tissue when effecting processing of sealing and welding, e.g., a part of a body cavity or a blood vessel of the living tissue and hence has an improved quality and reliability. Further, in Jpn. Pat. Appln. KOKAI Publication No. 2002-325772 is proposed an electric surgical apparatus which controls an output power or terminates processing in accordance with a change in an impedance of a living tissue based on an output time of a high-frequency power and a preset value of the impedance of the living tissue.

Bipolar type sealing forceps as one type of such apparatuses have a structure in which two distal ends (jaws) to which a high-frequency power is applied are configured into a double or single opening structure to hold a desired blood vessel or the like therebetween. These distal ends have insulating properties by using an insulating member such as ceramic or a resin to prevent an electrical short circuit from being generated between the distal ends when closed.

Since the two distal ends of the bipolar type sealing forceps respectively serve as electrodes, the forceps are used with a narrow inter-electrode distance as compared with monopolar type forceps. Therefore, when a living tissue or the like is held and a high-frequency power is applied, abnormal discharge (a spark) may occur between, e.g., edge parts of the two distal ends even in a state where the living tissue is interposed. This spark often continues when it once occurs, a fluctuation in a high-frequency power which is subjected to feedback control is also generated, and there occurs so-called chattering that impedance characteristics of a living tissue which usually rectilinearly or linearly vary fluctuate in a small range. As described above, chattering of an impedance involves a reduction in not only control over application of high-frequency power but also quality of processing, and makes a judgment upon termination of processing difficult.

### BRIEF SUMMARY OF THE INVENTION

The present invention provides an electric processing system which terminates abnormal discharge (extinguishes a spark) due to a high-frequency power, avoids a reduction in quality of sealing processing and can readily make a judgment upon end of processing.

The electric processing system comprises: bipolar type forceps which receive a high-frequency power applied from the outside and perform processing with respect to a living tissue held between distal ends; and a drive control apparatus which generates and outputs a high-frequency power which is intermittently applied to the forceps by feedback control which sets an output value of a subsequent high-frequency power based on the precedently output high-frequency power, and it further has in the drive control apparatus a control portion which samples the sequentially output high-frequency power, detects phases of a current and a voltage, detects occurrence of abnormal discharge between the distal ends of the forceps based on a phase difference of the current and the voltage, reduces a voltage of the high-frequency power and prolongs an application time.

Furthermore, the electric processing system comprises: bipolar type forceps which receive a high-frequency power which is applied from the outside and perform processing with respect to a living tissue held between distal ends; and a drive control apparatus which generates and outputs a high-frequency power which is intermittently applied to the forceps by feedback control which sets an output value of a subsequent high-frequency power based on the precedently output high-frequency power, and it further has in the drive control apparatus a control portion which samples a sequentially output high-frequency power, detects an impedance from a current and a voltage, detects occurrence of abnormal discharge between the distal ends of the forceps based on presence/absence of a pulsating signal produced in a differential signal obtained by differentiating a detection signal of the impedance, reduces a voltage of the high-frequency power and prolongs an application time.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing a configuration of an electric processing system in a first embodiment according to the present invention;

FIGS. 2A, 2B, 2C, 2D, 2E and 2F are views showing output voltage characteristics and impedance characteristics obtained by the electric processing system according to the first embodiment;

FIG. 3 is a flowchart illustrating feedback control over a high-frequency power in the first embodiment;

FIG. 4 is a view showing a configuration of an electric processing system in a second embodiment according to the present invention;

FIGS. 5A, 5B, 5C, 5D, 5E and 5F are views showing output voltage characteristics and impedance characteristics obtained by the electric processing system according to the second embodiment; and

FIG. 6 is a flowchart illustrating feedback control over a high-frequency power in the second embodiment.

### DETAILED DESCRIPTION OF THE PREFERRED EMBODIMENT

Embodiments according to the present invention will now be described in detail hereinafter with reference to the accompanying drawings.

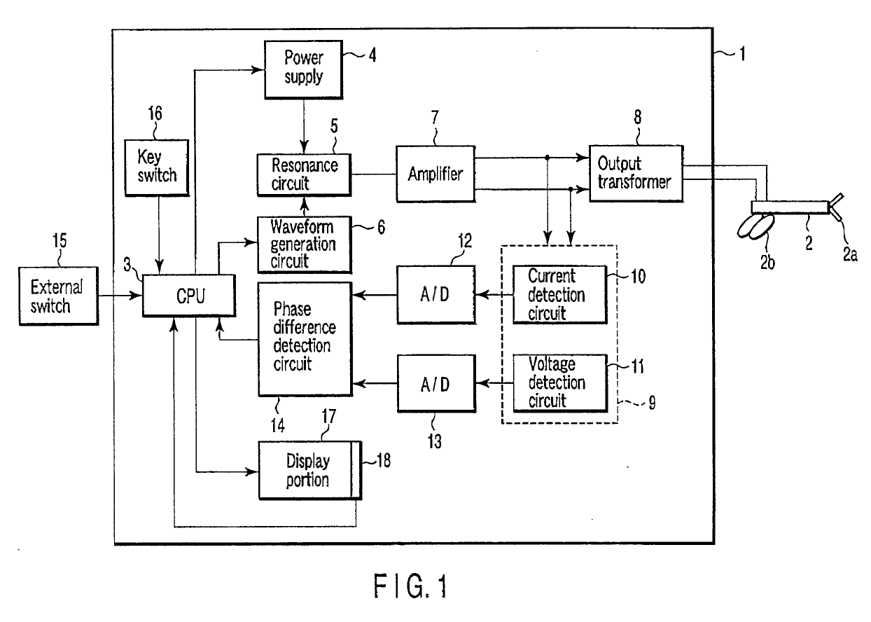

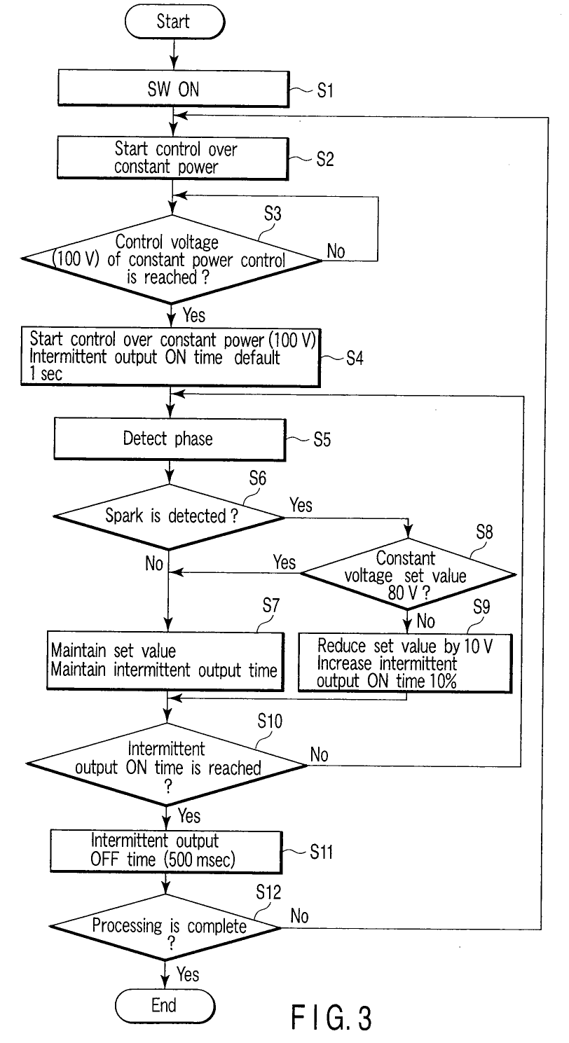

FIG. 1 shows a configuration of an electric processing system in a first embodiment according to the present invention. Moreover, FIG. 3 is a flowchart illustrating feedback control over a high-frequency power in this embodiment.

The electric processing system according to this embodiment is roughly constituted of a drive control apparatus main body 1 and an electric processing instrument, e.g., bipolar type sealing forceps 2 which perform welding processing with respect to a blood vessel or the like. Distal ends 2a in which two jaws to which a high-frequency power (a power value: a high-frequency current value×a high-frequency voltage value) can be applied are formed into a double opening or single opening structure are provided at the end of the sealing forceps 2a so that a living tissue or a blood vessel is held to perform processing such as incision, coagulation, sealing, welding or the like. An insulating member such as a ceramic member or a resin member is provided on each of the two jaws so that a non-energized condition can be attained in a closed state. Additionally, manipulating an operating portion 2b provided on an operator's hand side of the sealing forceps 2 can open/close the jaws of the distal ends 2a. It should be noted that a switch which turns application of the high-frequency power on and off may be provided in this operating portion 2b.

The drive control apparatus main body 1 is comprised of: a control portion (a CPU) 3 which controls the entire apparatus and performs feedback control over a high-frequency power applied to the sealing forceps 2; a power supply 4 which supplies a direct current; a resonance circuit 5 which converts the direct current into a high-frequency current (a high-frequency power); a waveform generation circuit 6 which controls a waveform of the high-frequency current generated by the resonance circuit 5; an amplifier 7 which amplifies the high-frequency power to a desired power value; an output transformer 8 which outputs the high-frequency power from the amplifier 7 to the jaws of the sealing forceps 2; a current/voltage detecting portion 9 consisting of a current detection circuit 10 which samples the output high-frequency power to detect a current value and a voltage detection circuit 11 which detects a voltage value; analog-to-digital converters 12 and 13 which respectively digitize the current value and the voltage value detected by the current detection circuit 10 and the voltage detection circuit 11; a phase difference detection circuit 14 which detects a change in a phase difference in detection values (the current value and the voltage value) converted into digital signals; an external switch 15 such as a hood switch which turns application of the high-frequency power on and off; a key switch (including a keyboard) 16 which is provided on the exterior of the apparatus and inputs operator instructions; a display portion 17 which displays an application state of the high-frequency voltage or information required for processing; and a touch panel switch 18 which is provided on a screen of the display portion 1 in order to input operator instructions like the key switch 16.

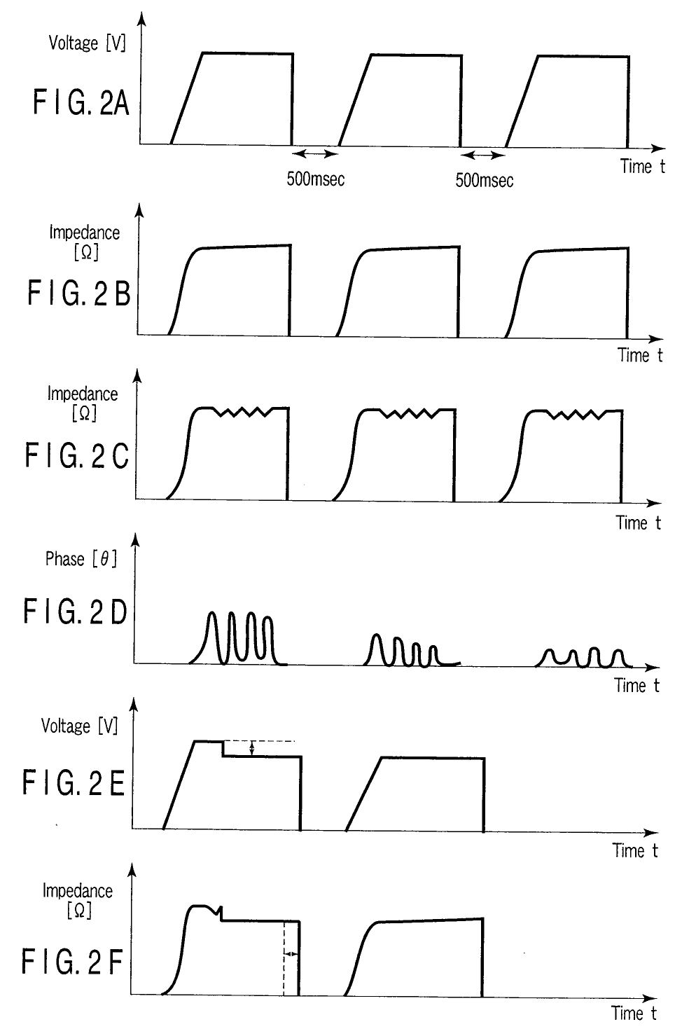

This drive control apparatus main body 1 applies a high-frequency power to the distal ends 2a of the sealing forceps 2 which hold a living tissue or a blood vessel therebetween. As shown in FIG. 2A, this high-frequency power is shaped into a pulse waveform based on an intermittent output timing that, e.g., an application time is approximately one section and a time interval of approximately 0.5 seconds is provided in such a manner that a temperature of a held living tissue or blood vessel and a temperature of a periphery of such a part are maintained at a predetermined proper temperature or below. An application state of this high-frequency power (an output value or an intermittent output timing) is appropriately changed in accordance with a design specification of the sealing forceps 2 or a state of a living tissue. An output value of the high-frequency power can be set by appropriately operating the key switch 16 or the touch panel switch 18.

It is sufficient to set an output waveform of this high-frequency power in such a manner that it becomes substantially equivalent to an amount of an applied energy based on a product of a sum of each application time of the intermittently applied high-frequency power and a high-frequency power (a high-frequency current value or a high-frequency voltage value). That is, when the high-frequency power is reduced, prolonging the application time to allow processing can suffice.

The phase difference detection circuit 14 detects a change in a phase difference in detection values (the current value and the voltage value) converted into digital signals. It is sufficient to detect a change in a phase difference between the current and the voltage to sense occurrence of abnormal discharge. It should be noted that a waveform of the high-frequency power generated in this embodiment is basically a sine waveform, and hence a zero cross circuit may be used to detect a phase difference.

FIGS. 2A to 2F show changes in a voltage waveform of the intermittently output high-frequency power, an impedance and a phase. Specifically, FIG. 2A shows a voltage waveform of a normal high-frequency power, and abnormal discharge (a spark) is not generated. FIG. 2B shows a change in an impedance of a living tissue or a blood vessel held between the distal ends 2a of the sealing forceps 2 to which the high-frequency power is applied. This change represents a normal state and corresponds to a change from several tens of ohms to approximately 500 Ω, and the impedance is increased in accordance with a change in the high-frequency power and then relatively gently increased. FIG. 2C shows a change in the impedance when abnormal discharge is produced. A value of the impedance calculated based on continuously generated sparks has chattering characteristics. FIG. 2D shows a phase detection signal detected in the phase difference detection circuit 14. FIG. 2E shows output characteristics of the high-frequency power whose voltage is reduced by feedback control of the CPU 3 at the time of generation of abnormal discharge. FIG. 2F shows a change in the impedance of a held living tissue or blood vessel involved by the high-frequency power whose voltage is reduced by feedback control of the CPU 3 at the time of occurrence of abnormal discharge.

A description will now be given as to output control of the thus configured electric processing system with reference to a flowchart of FIG. 3.

First, an operator turns on the external switch 15 to start processing (step S1). Based on this on operation, application of a high-frequency power to the sealing forceps 2 is started, and feedback control begins (step S2).

Then, a judgment is made upon whether a voltage value of the high-frequency power applied to the sealing forceps 2 has reached a preset control voltage (e.g., a constant voltage having an upper limit value of 100 V) (step S3). Voltage rising in this example has a waveform shape which is slightly inclined with respect to vertical rising of a regular pulse waveform as shown in FIG. 2 in order to avoid overshoot. It should be noted that a degree of inclination of rising is appropriately set in accordance with a design specification. In this manner, a factor of a reduction in quality of processing and start of abnormal discharge due to overshoot is eliminated in the waveform. The quality of processing described herein means realization of a sealed state without carbonation of a living tissue to be sealed or adhesion in a closely-attached state without rupture in the case of a blood vessel.

When the high-frequency power has not reached the control voltage yet (NO) in the judgment at step S3, the application state is maintained. On the other hand, when the high-frequency power has reached the control voltage (YES), the control voltage is maintained and an intermittent output is applied to the sealing forceps 2 in a pulse waveform with an application time of approximately one second (step S4). At this time, an intermittent time (an output stop time) is set to approximately 500 milliseconds. Of course, this output stop time is approximately set while considering a high-frequency power value in such a manner that a temperature of a held part and a temperature of its peripheral living tissue become less than a predetermined temperature.

Subsequently, a phase difference signal detected by the phase difference detection circuit 14 is detected from an output signal of the high-frequency power (step S5). This phase difference signal represents a phase difference between a high-frequency power to be input and an input high-frequency power with a change in an impedance, and is detected by the phase difference detection circuit 14. The CPU 3 uses this phase difference detection signal to judge whether abnormal discharge (a spark) has been generated in the sealing forceps 2 (step S6). When it is determined that abnormal discharge is not generated in this judgment (NO), application is continued with the set control voltage and intermittent output timing (step S7). On the other hand, when such a phase difference detection signal as shown in FIG. 2D is detected and it is determined that abnormal discharge (a spark) has been generated by the CPU 3 (YES), a judgment is made upon whether the current control voltage (a constant voltage) has been reduced to 80 V (a judgment reference value in this embodiment) (step S8). When it is determined that the control voltage (a constant voltage) is 80 V in this judgment (YES), this 80 V is maintained and the control shifts to step S7. Further, when the control voltage is not smaller than 80 V (NO), the control voltage is reduced by 10 V, and an application time is increased 10%. In this embodiment, in the case of the control voltage which is, e.g., 100 V, the control voltage is reduced to 90 V, and an application time of one second is prolonged to 1.1 seconds. Although a lower limit of the application voltage is set to 80 V in this embodiment, it is empirically set as a voltage which stops abnormal discharge, i.e., extinguishes a spark and enables appropriate processing. Therefore, the lower limit of the application voltage can be set to 80 V or above depending on electrical characteristics of sealing forceps 2 having a different design.

After the high-frequency power is applied at step S7, or after a reduction in the control voltage is set and the reduced voltage is applied at step S8, a judgment is made upon whether a preset application time (an intermittent output on time of approximately one second in this example) has been reached (step S10). If it is determined that the preset application time has not been reached in this judgment (NO), the control returns to step S5, and a change in an impedance is detected while maintaining application of the high-frequency power. On the other hand, if the preset application time has been reached (YES), output is stopped for the above-described output stop time of approximately 500 milliseconds (step S11).

Further, whether adhesion processing has been completed with respect to a blood vessel or the like is judged (step S12). As to a timing of completion of this adhesion processing, it is sufficient to use a known completion judgment method, e.g., an accumulated temperature (a sum of histories) of increases in temperature in a periphery of a held part, achievement of a preset impedance value at the time of completion or whether a phase difference has reached a specified value. If it is determined that welding processing has not been completed in the judgment (NO), the control returns to step S2 to continue the processing sequence. On the other hand, if the welding processing has been completed (YES), this processing sequence is terminated.

As described above, according to the first embodiment, a phase difference in the sequentially intermittently output high-frequency power is monitored for the feedback control over the high-frequency power applied to the bipolar type sealing forceps 2. When abnormal discharge (a spark) is produced at the distal ends, the high-frequency power is reduced and the application time is extended. As a result, the abnormal discharge can be terminated (the spark can be extinguished), a reduction in quality of processing can be avoided, and end of processing can be readily judged.

Furthermore, since abnormal discharge is avoided on the drive control apparatus main body 1 side in this embodiment, even if the sealing forceps 2 are changed over, the present invention can be applied by just changing a set value on the drive control apparatus main body 1 side, and the equivalent effect can be obtained, thereby providing high general versatility.

A second embodiment will now be described.

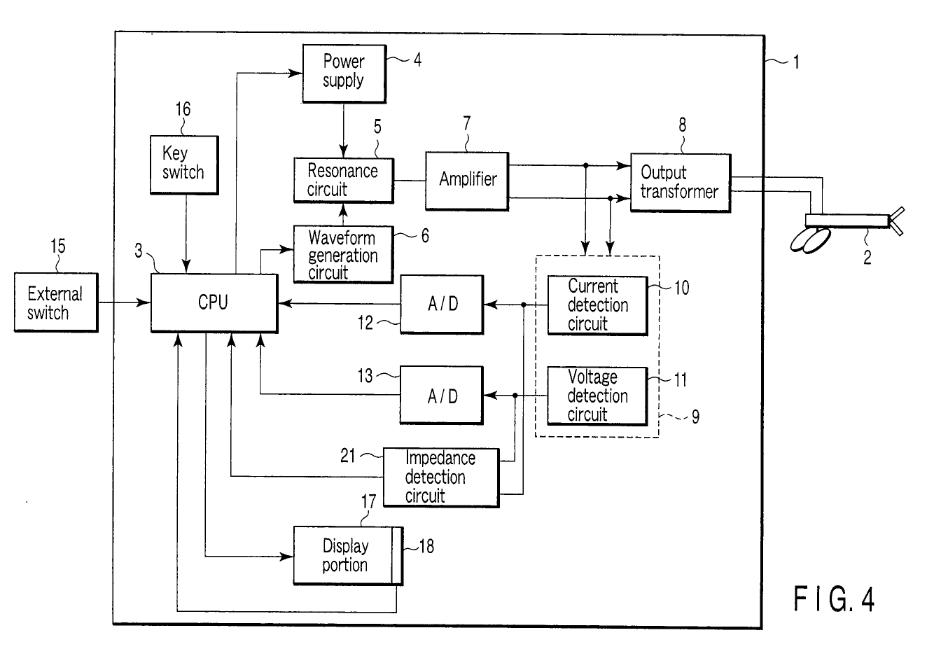

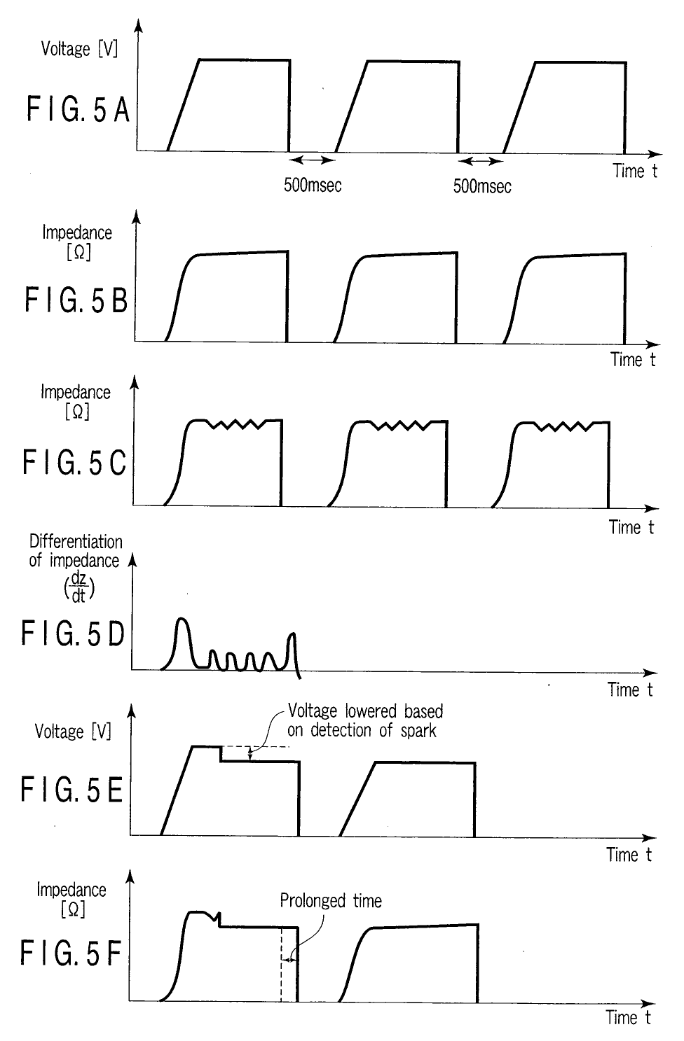

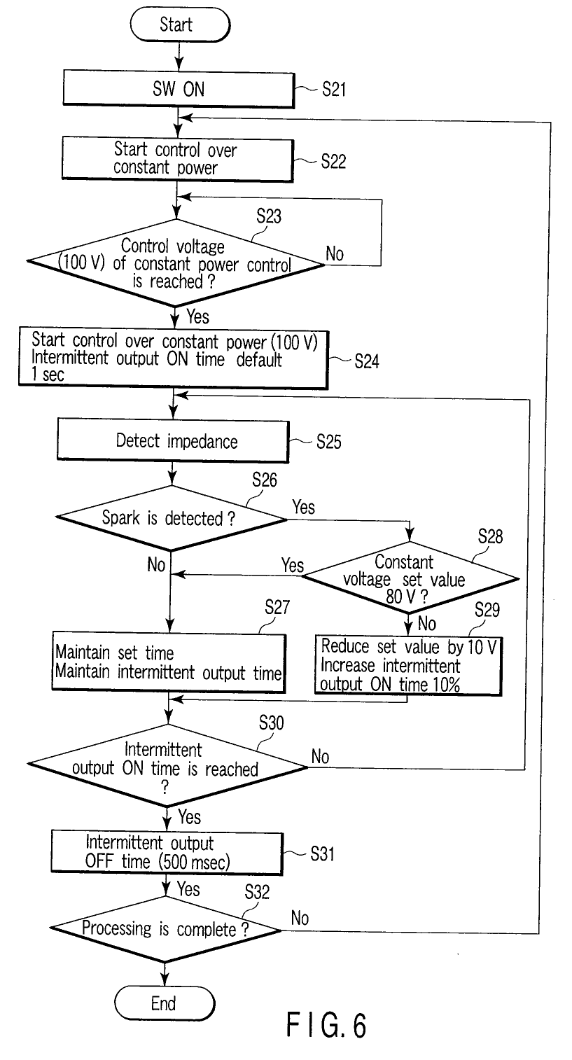

FIG. 4 shows a configuration of an electric processing system in the second embodiment according to the present invention. FIGS. 5 to 5F are views showing output voltage characteristics and impedance characteristics obtained by the electric processing system according to this embodiment. Moreover, FIG. 6 is a flowchart illustrating feedback control over a high-frequency power in this embodiment. Like reference numbers denote constituent parts in this embodiment which are equivalent to those in the first embodiment, thereby omitting a detailed description thereof.

The electric processing system according to this embodiment is constituted of a drive control apparatus main body 1 and an electric processing instrument, e.g., bipolar type sealing forceps 2 which perform welding processing with respect to a blood vessel or the like.

The sealing forceps 2 hold a living tissue or a blood vessel between distal ends 2a to carry out processing of incision and coagulation (sealing and welding) by using a high-frequency power. In this embodiment, a treatment concerning welding processing of a blood vessel or the like is performed. This embodiment provides a structure in which an impedance detection circuit 21 which detects a change in an impedance of a held living tissue based on a fluctuation in a high-frequency power is provided in place of the phase difference detection circuit 14 according to the first embodiment.

The drive control apparatus main body 1 is comprised of a control portion (a CPU) 3 which performs control over the entire apparatus and feedback control over a high-frequency power, a power supply 4, a resonance circuit 5 which generates a high-frequency power, a waveform generation circuit 6 which controls a waveform when generating a high-frequency power, an amplifier 7 which amplifies the high-frequency power, an output transformer 8 which outputs the high-frequency power to the sealing forceps 2, a current/voltage detecting portion 9 have a current detection circuit 10 and a voltage detection circuit 11, analog-to-digital converters 12 and 13 which respectively digitize a detected current value and voltage value, an impedance detection circuit 21 which samples the generated high-frequency power (a current value and a voltage value) to detect an impedance, an external switch 15 such as a hood switch, a key switch (including a keyboard) provided on the exterior of the apparatus, a display portion 17 which displays an application state of the high-frequency voltage or information required for processing, and a touch panel switch 18 which inputs operator instructions like the key switch 16.

This drive control apparatus main body 1 applies a high-frequency power to the distal ends 2a of the sealing forceps 2 which hold a living tissue or a blood vessel therebetween. As shown in FIG. 5A, this high-frequency power is shaped into a pulse waveform based on an intermittent output timing with an application time of approximately 1 second and a time interval of approximately 500 milliseconds in such a manner that a temperature of a periphery of a held living tissue or a blood vessel is maintained at a preset proper temperature or below. An application state of this high-frequency power is appropriately changed as in the first embodiment.

It is sufficient to set an output waveform of this high-frequency power in such a manner that it becomes substantially equivalent to an amount of an applied energy based on a product of a sum of each application time of the intermittently applied high-frequency power and a high-frequency power value (a high-frequency current value or a high-frequency voltage value). That is, when the high-frequency power value is reduced, it is sufficient to prolong an application time to enable processing.

The impedance detection circuit 21 according to this embodiment is constituted by using a differential circuit. In a differential output signal of an impedance which is output from this impedance detection circuit 21 and shown in FIG. 5D, a spark in abnormal discharge is represented as a pulsating signal having peaks generated between rising and falling (i.e., first and last parts in a section) peak parts of the high-frequency power However, since a high-frequency noise component is also included, using a filter to remove such a component is preferable. The impedance detection circuit 21 may be formed by using an inverse function arithmetic circuit or a logarithmic amplifier, or a divider circuit or the like can be used.

FIGS. 5A to 5F show changes in a voltage waveform of the intermittently generated high-frequency power and an impedance. Specifically, FIG. 5A shows a voltage waveform of a normal high-frequency power, and abnormal discharge (a spark) is not produced. FIG. 5B shows a change in an impedance of a living tissue or a blood vessel held between the distal ends 2a of the sealing forceps 2 to which the high-frequency power is applied. This change is equivalent to that in FIG. 2B. FIG. 5C shows a change in the impedance when abnormal discharge occurs. FIG. 5D shows a differential output signal of the impedance in the impedance detection circuit 21. FIG. 5E shows output characteristics of the high-frequency power whose voltage is reduced by feedback control of the CPU 3 at the time of occurrence of abnormal discharge. FIG. 5F shows a change in the impedance of a held living tissue or a blood vessel involved by the high-frequency power whose voltage is reduced by feedback control of the CPU 3 at the time of occurrence of abnormal discharge.

A description will now be given as to output control of the thus configured electric processing system with reference to a flowchart shown in FIG. 6. It should be noted that operations in steps S21 to S24 and S27 to S32 in the flowchart of this embodiment are the same as those at steps S1 to S4 and S7 to S12 in the flowchart of FIG. 3, and hence corresponding steps will be briefly explained.

First, an operator instructs start of processing to commence application of a high-frequency power to the sealing forceps 2 and to also begin feedback control (steps S21 and S22). A judgment is made upon whether a voltage value of the high-frequency power applied to the sealing forceps 2 has reached a preset control voltage (e.g., 100 V: an upper limit value) (step S23). In this embodiment, rising of the high-frequency power is slightly inclined to avoid overshoot, thereby eliminating a factor of a reduction in quality of processing and start of abnormal discharge due to overshoot.

When it is determined that the high-frequency power has not reached the control voltage in the judgment at step S23 (NO), this application state is maintained. On the other hand, when the high-frequency power has reached the control voltage (YES), this control voltage is determined as a constant voltage and applied to the sealing forceps 2 as an intermittent output having a pulse waveform with an application time of approximately one second (step S24). At this time, an intermittent time (an output stop time) is set to approximately 500 milliseconds. These settings are the same as those in the first embodiment.

Subsequently, a detection signal obtained by differentiating a change in an impedance in the sealing forceps 2 is detected from the output signal of the high-frequency power by the impedance detection circuit 21 (step S25). Whether abnormal discharge (a spark) has been generated in the sealing forceps 2 is judged based on this detection signal (step S26). If it is determined that abnormal discharge has not been generated in this judgment (NO), application is continued with the set control voltage and intermittent output timing (step S27). On the other hand, when a pulsating signal is detected in such a differential signal of the impedance as shown in FIG. 5D and it is determined that abnormal discharge (a spark) has been generated, whether the current control voltage (a constant voltage) has been reduced to 80 V is judged. In the case of 80 V, the control advances to step S27 to continue application. In the case of 80 V or above, the control voltage is reduced by 10 V and the application time of one second is increased 10% to be prolonged to 1.1 seconds as in the first embodiment (steps S28 and S29).

Then, after steps S27 and S29, a judgment is made upon whether the preset application time (an intermittent output on time of approximately one second in this example) has been reached (step S30). When it is determined that the present application time has not been reached (NO), the control returns to step S25 and a change in the impedance is detected while continuing application of the high-frequency power. On the other hand, when the preset application time has been reached (YES), the output is stopped for the above-described output stop time of approximately 500 milliseconds (step S11).

Moreover, a judgment is made upon whether welding processing has been completed with respect to a blood vessel or the like (step S12). As to a timing of completion of this welding processing, it is sufficient to use a known completion judgment method, e.g., a history of an increase in a temperature in a periphery of a held part or attainment of a preset impedance value at the time of completion. If it is determined that the welding processing has not been completed yet in this judgment (NO), the control returns to step S2 to continue the processing sequence. On the other hand, if the welding processing has been completed (YES), this processing sequence is terminated.

As described above, according to the second embodiment, a differential output of the impedance of the intermittently generated high-frequency power is sequentially monitored for the feedback control with respect to the high-frequency power applied to the bipolar type sealing forceps 2. When abnormal discharge (a spark) is produced at the distal ends, the high-frequency power is reduced and the application time is extended. As a result, the abnormal discharge can be terminated, a reduction in quality of processing can be avoided, and end of processing can be readily judged.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An electric processing system comprising:
bipolar type forceps which receive a high-frequency power applied from the outside and carry out processing to a living tissue held between distal ends;
a drive control apparatus which generates and outputs a high-frequency power which is intermittently applied to the forceps by feedback control which sets an output value of a subsequent high-frequency power based on the precedently output high-frequency power; and
a control portion which samples the sequentially output high-frequency power, detects a phase difference of a current and a voltage, detects occurrence of abnormal discharge between the distal ends based on the phase difference, reduces a voltage of the high-frequency power and prolongs an application time, the control portion being provided in the drive control apparatus.

2. The electric processing system according to claim 1,
wherein the voltage-reduced high-frequency power and the prolonged application time are set in a range that they become equivalent to an energy amount based on a product of a power value of the high-frequency power and the application time and in a range that a spark of the abnormal discharge is extinguished and desired sealing processing is carried out.

3. The electric processing system according to claim 1,
wherein the drive control apparatus comprises:
an output transformer which outputs the high-frequency power to the forceps;
an amplifier which amplifies a high-frequency power supplied to the output transformer;
a detecting portion which measures a voltage value and a current value in the high-frequency power input to the output transformer;
an analog-to-digital conversion portion which digitizes the detected current value and voltage value;
a phase difference detection circuit which detects a phase difference of the current value and the voltage value output from the analog-to-digital conversion portion;
a control circuit which detects occurrence of a spark between the distal ends of the forceps based on the phase difference, reduces a voltage of the applied high-frequency power to extinguish the spark, and prolongs an output time;
a waveform generation circuit which generates a specified output waveform based on an instruction from the control circuit; and
a power supply which generates the high-frequency power to be output based on an instruction from the control circuit.

4. An electric processing system comprising:
bipolar type forceps which receive a high-frequency power applied from the outside and carry out processing to a living tissue held between distal ends;
a drive control apparatus which generates and outputs a high-frequency power which is intermittently applied to the forceps by feedback control which sets an output value of a subsequent high-frequency power based on the precedently output high-frequency power; and
a control portion which samples the sequentially output high-frequency power, detects a tissue impedance from measuring an output current and an output voltage, detects occurrence of abnormal discharge between the distal ends of the forceps based on presence/absence of a pulsating signal produced in a differential signal obtained by differentiating a detection signal of the impedance, reduces a voltage of the high-frequency power and prolongs an application time, the control portion being provided in the drive control apparatus.
